(19) United States
(12) Patent Application Publication
(10) Pub. No.: US 2003/0022224 A1
(43) Pub. Date: Jan. 30, 2003

(54) Method of detecting binding reaction between protein and test substance

(75) Inventors: Hiroko Sakamoto, Tokyo (JP); Noriko Kato, Hachioji-shi (JP); Naoaki Okamoto, Hachioji-shi (JP)

Correspondence Address: Scully, Scott, Murphy & Presser, 400 Garden City Plaza, Garden City, NY, 11530-0299, US

(73) Assignee: OLYMPUS OPTICAL CO., LTD., TOKYO (JP)

(21) Appl. No.: 10/198,785

(22) Filed: Jul. 19, 2002

(30) Foreign Application Priority Data
Jul. 19, 2001 (JP) 2001-220444
Jul. 23, 2001 (JP) 2001-221963

(51) Int. Cl. C12Q 1/68; G01N 33/53; C12P 21/02; C12N 5/06

(52) U.S. Cl. 435/6; 435/7.1; 435/69.1; 435/325

(57) ABSTRACT

A method of detecting presence/absence of binding capacity of a test substance, with respect to a protein, comprising, (1) having a protein, which has been labeled with a fluorescence material, exist in a solution, and (2) while successively measuring fluorescence intensity from the fluorescent material, reacting the test substance with the fluorescence-labeled protein described in (1) above and determining presence/absence of binding capacity of the test substance, with respect to the protein, on the basis of the successive change in fluorescence intensity.

### CROSS-REFERENCE TO RELATED APPLICATIONS

This application is based upon and claims the benefit of priority from the prior Japanese Patent Applications No. 2001-220444, Jul. 19, 2001; and No. 2001-221963, Jul. 23, 2001, the entire contents of both of which are incorporated herein by reference.

### BACKGROUND OF THE INVENTION

1. Field of the Invention

The present invention relates to a method of detecting a binding reaction between a protein (a receptor, in particular) and a test substance. The detection method of the present invention is especially useful when a substance which acts as a ligand of a receptor and induces a signal is to be rapidly screened from a large amount of test substances.

The present invention also relates to a labeled protein to be used for said detection method, and a method of producing said labeled protein. The labeled protein of the present invention is produced in a solution by using an expression vector.

2. Description of the Related Art

In the study involved with analysis on protein functions, the main subject to be analyzed for study has conventionally been the sequence of a gene which encodes a protein. However, as a result of the rapid progress recently made in the analysis on human genome, the direction of the study involved with analysis on protein functions is now shifting, from the analysis of genes in human genome, to the analysis on kinetics and function of proteins encoded by such genes.

In vivo, a variety of molecules (e.g., functional proteins such as enzymes and receptors; proteins for holding a cell structure; and biologically active molecules such as lipids, sugar chain proteins and ions) having specific functions are constantly moving and changing in accordance with a variety of gene expression and signal transduction, and the life mechanism is maintained by such movement and change.

Above all, it has recently been revealed that various response reactions, which occur in vivo, responsive to stimulus from the outer environment are carried out by signal transduction by way of receptor-ligand binding reaction in a cell. Receptors are mainly classified into two groups: those present on a cell membrane; and those present inside a cell nucleus. In particular, the group of receptors which are present inside a cell nucleus and called “a nuclear receptor”, specifically bind low-weight molecules such as hormone and vitamins as ligands, and directly controls the transcription processes of various genes. Therefore, a compound which can be bound to a nuclear receptor is expected to have various pharmaceutical effects directly in vivo. Accordingly, searching such a compound in an efficient manner is an important object in developing new drugs.

In recent years, some chemical substances which are present in the environment and have undesirable influence, such as abnormal development of genital organs, on an organism (i.e., endocrine disrupting chemicals) have been found seriously problematic. Most of the endocrine disrupting chemicals function as ligands specifically bound to nuclear receptors, and generate a false signal which is irrelevant to the “correct” ligands inherent to the organism, thereby causing bad influences on the organism. In particular, approximately 90% of the known endocrine disrupting chemicals allegedly function as ligands of the estrogen receptor, which is one of the nuclear receptors. In the situation as described above, the necessity of screening a numerous number of known chemical substances by utilizing the capacity thereof of serving as a ligand of the estrogen receptor (i.e., the capacity thereof of being bound to the estrogen receptor) has been recognized, and a large-scale screening is now being performed in such a manner all over the world.

In order to accurately observe and analyze the kinetics of a targeted protein (such as a receptor protein as described above) in vivo, it is necessary to observe the kinetics, at the molecular level, of the protein whose three-dimensional structure, in which the inherent functions of the protein can be fully demonstrated, has been maintained. As means for achieving this object, a method of labeling the targeted protein with a label so that the labeled protein can be traced, is generally employed in the prior art.

For example, one of the conventional methods of labeling the targeted protein is a fluorescence-labeling method (Cytometry Dec. 1, 2000; 41 (4): 316-20: Lipopolysaccharide (LPS) labeled with Alexa 488 hydrazide as a novel probe for LPS binding studies. Triantafilou K, Triantafilou M, Fernandez N.). This is a method of having a protein non-specifically chemically bonding with a fluorescent material such as FITC (i.e., fluorescein isothiocyanate) and Alexa. However, in the case of the fluorescence-labeling method as described above, when an interaction between a protein and another substance (e.g., a receptor-ligand interaction and an antigen-antibody interaction) is analyzed, the three-dimensional structure of the targeted protein is often subjected to variation and thus accurate analysis results may not be obtained.

Another conventional method, which is more excellent in specificity and sensitivity than the aforementioned fluorescence-labeling method, is a method of producing the targeted protein by using a gene-introduced cell. For example, a method of using a green-fluorescent protein (which protein will be referred to as “GFP” hereinafter) as a label is disclosed in “Biotechniques” 1995 October; 19 (4): 650-5 Related Articles, Books Green fluorescent protein as a reporter of gene expression and protein localization. Kain S R, Adams M, Kondepudi A, Yang T T, Ward W W, Kitts P.). In this method, a gene encoding the targeted protein and a gene encoding GFP are incorporated to the same vector, the resultant recombinant vector is introduced into a cell, and a fused protein of GFP and the targeted protein is expressed, whereby the targeted protein which has been labeled is obtained. Here, GFP used as the label is a biomolecule derived from Aequorea victoria and is already known to have no biotoxicity. The binding of GFP to the targeted protein can be selectively carried out with respect to the terminal end of the protein. Accordingly, by employing the technique as described above, the kinetics of the targeted protein can be observed while the three-dimensional structure thereof is phisiofunctionally maintained.

The kinetics, inside a cell, of the targeted protein which has been fluorescent-labeled by the aforementioned method is observed by generally using a phase-contrast microscope or a differential interference microscope. However, the resolving power of these microscopes is no better than 0.2 to 0.3 μm or so. Such poor resolving power may be somehow tolerated when a micro-structure inside a living cell is observed as an image, but is not acceptable for minute analysis of interactions between a protein and another substance.

Further, when the nuclear receptor is the targeted protein and the kinetics thereof is observed, problems specific to this case will arise. It has been understood, from the analysis in the past, that a nuclear receptor generally functions as follows in vivo (refer to FIG. 1). First, the nuclear receptor receives a specific ligand, thereby forming the receptor/ligand complex (Stage I), and the complexes dimerize. The dimer recognizes a specific base sequence (a receptor-responsive sequence) of the intranuclear DNA and is bound thereto, thereby forming a receptor/DNA complex (Stage II). The receptor/DNA complex is activated by a coactivator present inside the nucleus, facilitates transcription of a gene existing at the downstream side, and induces various bioactivity (Stage III).

Most of the conventional ligand-screening methods effect detection at the initial stage at which the nuclear receptor functions, i.e., detect binding of a receptor and a ligand. The technique called “receptor-binding assay” is most commonly carried out. In the receptor-binding assay, a labeled ligand of a known type and a non-labeled test substance are at first competitively reacted with the targeted receptor. Thereafter, the labeled ligands which have not been bound to the targeted receptor are removed by washing, and the amount of the labeled ligands which have been bound to the targeted receptor is measured. By measuring the amount of the labeled ligands which have been bound to the receptor in such a manner, absence/presence of the capacity, of the test substance, of binding itself to the targeted receptor, as well as the amplitude of the capacity, are detected. This method is simple and allows easy arrangement of experiment systems. However, as a special facility is required when a radioisotope is used as a label, and as a washing process is required prior to measuring, the method does not satisfy the requirements essential in high-throughput screening. Further, as labeled ligands of a known type need to be added as a tracer substance to the reaction system, the method cannot be employed for assaying a receptor whose inherent ligand has not be known (an orphan receptor, for example). This could be a serious disadvantage in the case of developing a new drug, in particular, because a ligand with respect to an orphan receptor quite often turns out to be effective as a new pharmaceutical drug.

The intracellular kinetics of a targeted protein can also be analyzed by using Fluorescence Correlation Spectroscopy (which will be referred to as “FCS” hereinafter) which has been developed in recent years. FCS is a technique in which the fluctuation movement, in the medium, of the targeted molecules which have been labeled with fluorescence is measured in the minute confocal area, which is in the order of f (10⁻¹⁵) L and provided in a reaction solution by laser radiation, whereby the micromovement of the individual targeted molecules is measured by using an autocorrelation function. By using the method described above, the physical quantities such as the number and the size of the targeted molecules present in the measuring system can be obtained. On the basis of such measurements, the state of the targeted molecules present in the system is measured continuously, thereby the interaction between the targeted protein and another molecule can be analyzed in detail.

As a specific example of using FCS, there has been a report of a method of fluorescence-labeling a test substance itself and having, the labeled test substance, reacted with the targeted receptor (PCT National Publication No. 11-502608: “Method of/Device for evaluating the degree of adaptation of biomacromolecules”). According to this method, presence/absence of binding between the test substance and the targeted receptor is detected by measuring the diffusion time, in the reaction solution, of the test substance before and after the reaction. Specifically, the test substance is fluorescence-labeled in advance, and the labeled test substance is added to the targeted receptor present in a cell or a test tube, thereby having the substance reacted with the targeted receptor. In a case where the test substance is bound to the receptor, as the test substance, having a relatively low molecular weight, forms a complex with a receptor protein having a relatively large molecular weight, the apparent molecular weight of the substance increases. As a result, the rate at which the substance diffuses in the solution is expected to be slowed. The binding property, of the test substance, with respect to the receptor is detected on the basis of the diffusion time.

According to this method, separation/removal, from the reaction solution, of the labeled substance which has not been reacted is not required. Addition of a labeled ligand of a known type as a tracer is not required, either. Further, the diffusion time of the fluorescence-labeled molecules in the reaction solution can be measured in a few seconds by using FCS. In these aspects, this method satisfies requirements as a high-throughput detection system. However, this method necessitates fluorescence-labeling of the test substance. Therefore, in a case where test substances of various types are to be screened, fluorescence-labeling all the test substances, without causing any influence on the molecular structure, will probably be enormously difficult and costly. Moreover, in the method, in order to detect presence/absence of the binding capacity with clear distinction therebetween, it is essential to remove the unreacted fluorescence reagent, as much as possible, which generates at the time of fluorescence-labeling of the test substance, so that the background noise is suppressed. In consideration of such difficulties and the amount of labor, the method clearly has an aspect which is not suitable for high-throughput screening.

Further, as an example of biological application of FCS described above, there has been a report of a method of FCS measurement in which GFP introduced into a living cell is measured (Proc. Natl. Acad. Sci. USA vol. 96, pp. 10123-10128, 1999). The aforementioned reference points out that association of GFP with intracellular molecules of plural types of size occurs even in a stationary state in which no signal has been received. In other words, according to the conventional FCS method, it is extremely difficult to measure, in a stable manner, the targeted molecules in a state in which the molecules are present inside a cell (that is, it is very difficult to achieve accurate analysis by FCS).

As yet another method, there is a method which has recently been developed, in which the test substance is reacted with the targeted receptor which has been brought into the solid-phase on a sensor, so that the direct, intermolecular interaction between the receptor and the test substance is detected by the surface plasmon resonance or mass spectrometry. This method necessitates neither addition of ligand of a known type nor labeling of the test substance. However, this method has aspects which are not suitable for high-throughput screening, such as the washing process of separating/removing the unreacted substance being required, the chip in which the targeted receptor has been brought into the solid phase being expensive, and the like.

Examples of yet another screening method include: the yeast two-hybrid method in which dimerization between the receptors is detected by luminescence; and a reporter assay in which the final stage of the nuclear receptor functioning (i.e., the stage at which the receptor/ligand complex is bound to the targeted DNA sequence, so that the expression of the downstream gene is induced) is detected by luminescence. However, each of these methods needs a living organism such as yeast, cultured cells or the like as the material and is costly and time-consuming. In short, these methods are not suitable for high-throughput screening.

As described above, any of the conventional methods has problems to be solved, when the method is used as a method of detecting whether or not the test substance can be bound to a protein (mainly a receptor).

### BRIEF SUMMARY OF THE INVENTION

In consideration of the problems described above, one object of the present invention is to provide a method of detecting a binding reaction between a protein (mainly a receptor) and a test substance, which method is suitable for high-throughput detection and allows stable and accurate detection. Particularly, an object of the present invention is to provide a method which allows rapid and accurate analysis of action of a test substance on a hormone receptor.

Specifically, an object of the present invention is to provide a method which allows rapid and accurate detection of action of a test substance on a hormone receptor, by utilizing measurement of fluorescent molecules by FCS and stably analyzing the kinetics of the fluorescence-labeled molecules. More specifically, an object of the present invention includes: accurately detecting binding, of a test substance, to a protein by adding a nucleic acid to a system in which the binding reaction between the protein (mainly a receptor) and the test substance is detected. In addition, an object of the present invention includes: labeling the biomolecules to be analyzed with a fluorescent material, in a stable manner; reducing meaningless association of the lebeled biomolecules with intracellular molecules, thereby reducing noise; and constructing the labeled biomolecules in a solution in a stable manner, thereby providing a simple and highly reproducible detection system.

Another object of the present invention is to provide a labeled protein produced in a solution by using an expression vector, for the use in the aforementioned method, and to provide a method of producing the labeled protein.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

FIG. 1 is a view showing the operation mechanism of a nuclear receptor;

FIG. 2 is a view showing a preferable example of a Fluorescence Correlation Spectroscopy (FCS) device;

FIG. 3 is a view showing a reaction solution model in which a test substance is not capable of acting as a ligand of a receptor;

FIG. 4 is a view showing a reaction solution model in which a test substance is capable of acting as a ligand of a receptor;

FIG. 5 is a view showing a structure of a complex constituted of a receptor, a test substance and a DNA fragment;

FIGS. 6A and 6B are views showing a vector map of a vector used in one embodiment of the present invention, respectively;

FIG. 7 is a photograph showing the results of electrophoresis performed for digested fragments of a pSPORT1 vector, a fusion gene of (GFP+ERβ), and an ERβ gene, respectively;

FIGS. 8A and 8B are electrophoresis photographs each showing the results of PCR performed for a (GFP+ER) clone and an ER clone;

FIG. 9 is an electrophoresis photograph of a fragment obtained by digesting a (GFP+ER) clone with a restriction enzyme;

FIG. 10 is an electrophoresis photograph of a fragment obtained by digesting an ER clone with a restriction enzyme;

FIG. 11 is an electrophoresis photograph of a product obtained in the in vitro expression system; and

FIG. 12 is a graph showing the results of endocrine disrupting chemicals-detection by using GFP-ER obtained by in vitro transcription.

In the drawings, each reference numeral represents a corresponding component as follows.

1. Laser source

2. Means for regulating laser intensity

3. Means for selecting laser attenuation rate

4. Dichroic mirror

5. Object lens

6. Stage

7. Filter

8. Tube lens

9. Reflecting mirror

10. Pinhole,

11. Lens

12. Photodetector

13. Means for recording fluorescence intensity

14. Means for detecting attenuation rate of fluorescence intensity

21. Fluorescence-labeled receptor

21a. Receptor

21b. Fluorescent material

22. Test substance

23. DNA fragment having receptor-responsive sequence

24. Receptor/test substance complex

25. Receptor/test substance/DNA fragment complex

### DETAILED DESCRIPTION OF THE INVENTION

In consideration of the aforementioned problems to be solved by the present invention, the inventors of the present invention have reached an idea that it is essential to extract the targeted protein from a cell and have the protein exist in a solution in an homogeneous state, in order to construct detection system in which meaningless association of the targeted protein with intracellular molecules does not occur. As a result of the assiduous study on the basis of the idea, the inventors of the present invention made it possible to produce a fused-protein of a fluorescent protein and a targeted protein (e.g., a hormone receptor) in vitro, by using in vitro translation/expression (cell-free translation method) as one method of genetic engineering. By using the fused protein obtained as described above, the inventors of the present invention have succeeded in creating a pure and homogeneous state of solution in which impurities which could cause association as in a cell are substantially eliminated, thereby solving the aforementioned problems.

Further, a method of the present invention, which is suitable for high-throughput detection, needs to satisfy the following requirements.

(1) Presence/absence of the binding capacity of a test substance with respect to a receptor can be detected with clear distinction between presence/absence of the capacity.

(2) The binding property of the test substance with respect to receptors of various types can be easily determined.

(3) No complicated or troublesome operations such as separation/washing of the materials added in the assay system are required in the process prior to detection.

(4) Time required for detection is relatively short.

(5) The reagents used for the assay are easily available, inexpensive and easy to handle.

(6) Assay can be conducted sufficiently, if the amount of the obtained substance is extremely small (it often happens in the field of creating a new drug, in particular, that the test substance can be obtained only by an extremely small amount in the production/synthesis process).

In the present invention, “high-throughput detection” represents that, for a large number of test substances of the same or different types, determination of presence/absence of a reaction derived from each test substance or determination of the amount of the reaction is carried out in a short time. A “high-throughput detection system” represents all of the steps required for carrying out the “high-throughput detection”. “High-throughput screening” represents that, after carrying out the “high-throughput detection”, the case where a reaction caused by a specific substance has been confirmed or the case where the amount of a reaction has exceeded a predetermined amount are selected in a short time from a numerous number of results including other cases. Further, a “high-throughput test” represents any test in which test results are obtained by carrying out the “high-throughput detection”.

In order to solve the aforementioned problems, the inventors of the present invention have made a research as described below. The scheme thereof is as follows. First, in order to carrying out high-throughput screening for a relatively large amount of the test substances, the method of the present invention has a presupposition that, as described above, receptor molecules suspended in a solution are employed in stead of receptor molecules expressed inside cells.

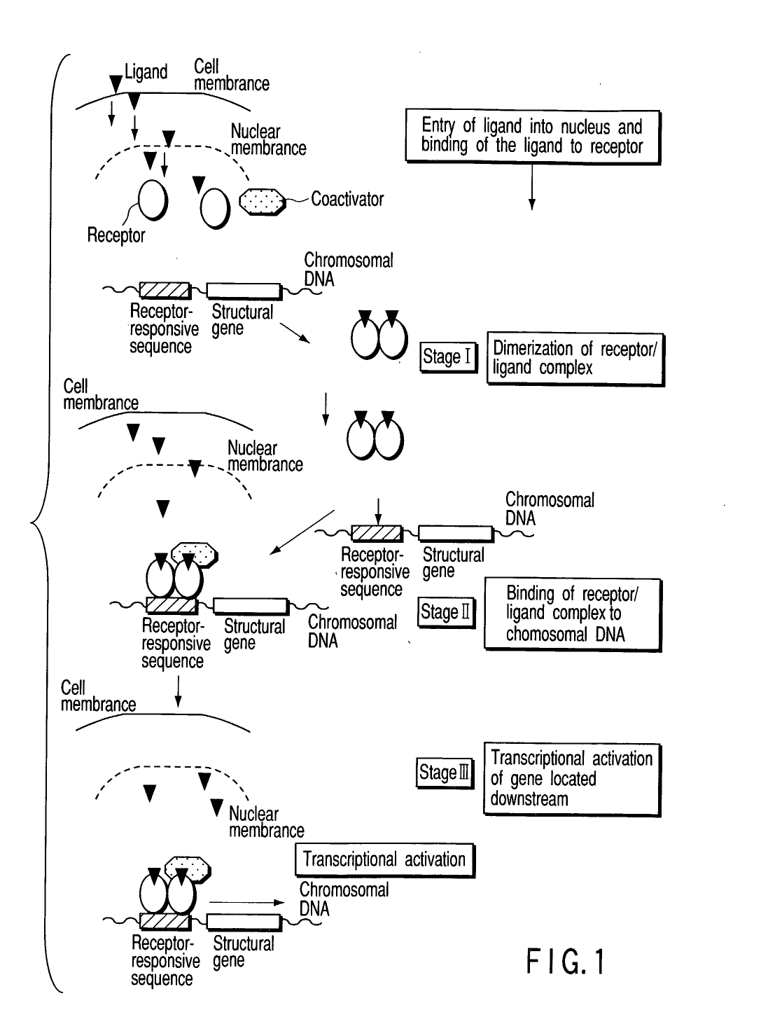

In a case where the protein is a nuclear receptor, the nuclear receptor receives a ligand and forms a dimer, as shown in FIG. 1 (Stage I). Thereafter, the receptors (that is a dimer) are bound to the targeted DNA sequence (Stage II), and causes transcriptional activation of the downstream gene (Stage III). Here, if the binding reaction of the receptor with the test substance is detected on the basis of difference in diffusion time of the molecules, it is assumed that such detection is possible at either Stage I where the receptors form a dimer or Stage II where the receptors and DNA form a complex. However, it is theoretically expected that the difference in diffusion time of the molecules will be larger when the binding reaction of the receptor with the test substance is detected on the basis of the difference in diffusion time between the receptor monomer and the receptor/DNA complex (i.e., at Stage II), than when the binding reaction of the receptor with the test substance is detected on the basis of the difference in diffusion time between the receptor monomer and the dimer thereof (i.e., at Stage I). Therefore, the inventors of the present invention have newly turned their attention to the point that the binding reaction of the receptor with the test substance can be detected at Stage II with clearer distinction between positive/negative than is at Stage I.

In the conventional receptor-ligand binding assays, the majority of these assays only detect presence/absence of binding of the receptor with the test substance or the binding affinity therebetween, and hardly determine the binding affinity of the receptor/ligand complex with respect to the targeted DNA sequence at the later stages of the reaction. However, there is a report which has recently been made that the intensity of the activity of the test substance, as a ligand, is more closely correlated with the affinity of the receptor/test substance complex with respect to the targeted DNA sequence, rather than the affinity of the test substance with respect to the receptor. (U.S. Pat. No. 5,888,738: Design of Drugs Involving Receptor-Ligand-DNA Interactions).

In consideration of the content as discussed above, the inventors of the present invention have found means for solving the aforementioned problems.

According to one aspect of the present invention, there is provided a method of detecting presence/absence of binding capacity of a test substance to a protein, comprising the steps of:

(1) having a protein, which has been labeled with a fluorescent material, exist in a solution; and

(2) while successively measuring the intensity of fluorescence emitted from the fluorescent material, reacting the test substance with the labeled protein described in (1), thereby determining presence/absence of binding capacity of the test substance with respect to the protein, on the basis of the measured change in fluorescence intensity. This aspect of the present invention will be referred to as “first embodiment” hereinafter.

According to another aspect of the present invention, there is provided a method of detecting a binding reaction of a receptor and a test substance, comprising the steps of: (1) maintaining a receptor which has been labeled with a marker capable of generating a light signal, a test substance, and a fragment of nucleic acid containing a specific nucleic acid sequence to which a receptor/ligand complex can be bound, in a solution in which the receptor and the ligand can form a complex and the receptor/ligand complex can be bound to the specific nucleic acid sequence; and (2) detecting presence/absence of a receptor/test substance/nucleic acid fragment complex, which is formed as a result of the receptor and the test substance forming a complex and this complex being bound to the fragment of the nucleic acid. This aspect of the present invention will be referred to as “second embodiment” hereinafter.

The first embodiment and the second embodiment can be combined in an appropriate manner, unless such combination causes any significant conflicts in technological terms.

Hereinafter, the first embodiment and the second embodiment will be described in detail in the order. It should be noted that the description of the first embodiment and that of the second embodiment might be referred to each other in an appropriately combined manner, unless such combination causes any significant conflicts in technological terms.

<First Embodiment>

1. Summary

According to a first embodiment of the present invention, a method of detecting presence/absence of binding capacity, of a test substance, with respect to a protein is provided.

The term “protein” herein represents, for example, a protein as a hormone receptor, an antigen, an antibody or the like having a characteristic of being specifically bound to a specific substance. Accordingly, the present invention is preferably used for detecting such a substance as is specifically bound to a protein. Although an embodiment in which a hormone receptor is used as an example of proteins will be described hereinafter, the present invention is not restricted to a hormone receptor and can be similarly implemented for other proteins.

In the present invention, the protein is preferably a protein which should naturally exist in a cell. Accordingly, in the present invention, the reaction of a protein with a test substance can be proceeded in a solution, by having the targeted protein (either collected from a cell or artificially synthesized) exist in an appropriate solution. As proteins, which should not naturally exist in a solution, are reacted in a solution, the conditions required for causing the binding reaction and measuring the result of the reaction can be easily made preferable, so that such a method using a solution system can be applied to the desired measuring method. If the binding reaction of the protein with the test substance is to be successively monitored and measured, it will be convenient to measure the change caused by the binding reaction according to a morphological or kinedynamic method. In a case where both the protein and the test substance have explicit morphological characteristics, a morphological method can be used. However, as most of the binding reactions are involved with extremely minute morphological change, measurement of kinedynamic change is preferable in terms of enhancing measurement precision.

2. Method of detecting presence/absence of action, of test substance, on hormone receptor

The “test substance”, which is measured according to the present embodiment, represents a chemical substance which is suspected of acting on the targeted hormone receptor and is other than the “true” or intrinsic ligand. Accordingly, any suspicious chemical substances which might be endocrine disrupting chemicals can be tested according to the method of the present invention.

The term “an action of the test substance, on the hormone receptor” used in the present specification represents an action of the test substance, on the hormone receptor, which action is similar to the physiological action caused to the hormone receptor by the “true” ligand. Examples of such an action include: the test substance being bound to the ligand-binding region of the targeted hormone receptor i.e., the test substance exhibiting affinity with respect to the ligand-binding region of the targeted hormone receptor; and the test substance, which has been bound to the receptor, having an influence on the state in which the hormone receptor exists. Here, the action that “the test substance has an influence on the state in which the hormone receptor exists” indicates that the size of one molecule of the receptor changes as a result of the binding of the test substance to the receptor. For example, the action includes: a test substance being bound to a hormone receptor which normally exists as a monomer and the hormone receptor being dimerized.

The term “in vitro” used in the present specification is employed for convenience in the description and represents, in particular, an in vitro assay system which does not always necessitate cells or which includes no cells (i.e., which is cell-free). Accordingly, the term “in vitro” dose not represent “inside of a test tube”, which is the literal meaning thereof. The detection method and the production method according to the present invention can be implemented in any containers including a test tube, a beaker, a microtube, a multi-well plate and the like or even on a plate which is capable of retaining the necessary amount of liquid. In the present specification, the term “in a solution” is virtually synonymous with the term “in vitro”.

According to the present embodiment, the detection method can be carried out by using any measuring method in which the intensity of fluorescence emitted from the protein which has been labeled with a fluorescent material is successively measured, so that presence/absence of binding capacity of a test substance to a protein can be determined on the basis of presence/absence of change obtained when the test substance is reacted with the protein or on the basis of the magnitude of the change. Preferably, the detection method can be carried out by using Fluorescence Correlation Spectroscopy, which will be referred to as “FCS” hereinafter. Specifically, a hormone receptor which has been labeled with a fluorescent material is brought into a state in which the hormone receptor exists in a solution and a test substance is reacted with the labeled hormone receptor, so that the state of the molecule (i.e., the hormone receptor) present therein is analyzed by FCS.

3. Fluorescence-labeled hormone receptor

The fluorescence-labeled hormone receptor used in the present invention is any hormone receptor which has been labeled with a fluorescent material.

The hormone receptor used in the present invention may be any of a receptor present in a cell membrane, a receptor present in cytoplasm and a receptor present in the nucleus. A receptor present in cytoplasm is preferable and a receptor present in a nucleus is particularly preferable.

Examples of the preferable hormone receptor include proteins which belong to the nuclear hormone receptor superfamily, such as the estrogen receptor, the progesterone receptor, the thyroid hormone receptor, the glucocorticoid receptor and the like. The especially preferable receptor is the estrogen receptor, which includes human estrogen receptor a (which will be also referred to as “hERα” hereinafter) and human estrogen receptor β (which will be also referred to as “hERβ” hereinafter).

As described below, the FCS technique is a technique in which a desired analysis is carried out on the basis of a change in the size of the molecules. Accordingly, in the present invention, it is preferable that the size of the molecules of the hormone receptor change between before and after the binding of the “true” ligand or the test substance to the hormone receptor. For example, a hormone receptor whose molecular size is changed (e.g., increased) as a whole after the binding of the ligand or the test substance thereto, or a hormone receptor which exists as a monomer before the binding of the ligand thereto and then is dimerized or polymerized by the action of the ligand or the test substance after the binding reaction, are especially preferable.

In the case of the estrogen receptor, the estrogen receptor forms a dimer when a ligand is bound thereto. The activity of estrogen in a cell under the physiological condition are aroused as follows: estrogen is bound to the estrogen receptor, whereby a complex is formed; a dimer is formed by two complexes; the dimer is bound to an estrogen-responsive sequence located at the upstream side of the targeted gene; and a coactivator is then bound to the dimer, whereby the genetic product is transcribed and translated. As described above, it is particularly preferable that the hormone receptor such as estrogen, which receptor is dimerized in the expression process of the action of the hormone, is used according to the method of the present invention.

The fluorescent material for labeling the hormone receptor protein used in the present embodiment preferably exists as a protein and generates a light signal including fluorescence and luminescence by laser irradiation or the energy of its own. When the fluorescent material is collected from the nature, the material may be either GFP extracted from Aequorea victoria or YFP, CFP, RFP as modifications thereof. Alternatively, the fluorescent material may be a fluorescent protein similar to GFP and the like, which is derived from Renilla mulleri (sea pansy). Examples of such a fluorescent protein include Green Fluorescent Protein (which will be referred to as “GFP” hereinafter), Cyan Fluorescent Protein (which will be referred to as “CFP” hereinafter), Yellow Fluorescent Protein (which will be referred to as “YFP” hereinafter) and Red Fluorescent Protein (which will be referred to as “RFP” hereinafter). Each of these fluorescent proteins can be expressed as a fused protein in which the fluorescent protein has been fused with a hormone receptor protein, by using DNA fragments having gene sequences encoding these proteins and employing the known gene recombination technique as described below.

4. Method of producing fluorescence-labeled hormone receptor

A fluorescence-labeled hormone receptor used according to the present invention is produced in vitro, by using an expression vector constructed by utilizing the gene recombination technique.

The conventional GFP vector has a mechanism in which the promoter sequence, for transcriptionally activating the targeted protein-coding gene (which has been inserted in the vector), functions only in a cell. When such a conventional GFP vector is used, the vector is genetically introduced to a mammal cell or a microorganism, and the RNA transcriptional activation enzyme of the host is utilized. The inventors of the present invention have introduced a RNA promoter necessary for the synthesis of the RNA transcriptional activation enzyme, at the upstream side of the targeted protein-coding gene that has been inserted in the fluorescent protein vector. By having, the expression vector obtained in such a manner, coexist with the corresponding enzymes in an appropriate cell-free system, the gene in the expression vector is transcriptionally activated, whereby the RNA synthesis is enabled and the targeted protein is produced.

The vector used in the present invention is a vector having a RNA polymerase promoter sequence. Further, the promoter used in the present invention is, for example, a promoter for a RNA polymerase such as T3, T7 or SP6.

When the targeted, fluorescence-labeled hormone receptor is synthesized in vitro, an expression vector, to which the targeted gene has been introduced, is first constructed. For example, a gene which codes a fluorescent protein and a gene which codes a targeted hormone receptor may be incorporated to a vector containing a promoter sequence for expressing the desired genes in the cell-free condition and a restriction enzyme site which can be used for gene introduction.

Specifically, an expression vector can be constructed by first preparing a fusion gene of a gene coding a fluorescent protein and a gene coding the targeted hormone receptor and incorporating the fusion gene to a desired vector. For example, a gene coding the targeted hormone receptor protein is fused at the downstream side of the GFP gene, and it may be incorporated to pSPORT1 including a T7RNA promoter sequence (manufactured by Lifetech Oriental co., plasmid pSPORT1).

The fusion of the gene coding the aforementioned hormone receptor, with the gene coding the fluorescent protein, can be carried out, for example, by first cloning the gene of the targeted hormone receptor and then effecting ligation, in the expression vector, of the receptor gene with the fluorescent protein gene incorporated in advance in the expression vector.

In order to effect such ligation, it is effective to employ any of multi-cloning site sequences. This sequence enables digestion by a restriction enzyme and ligation by ligase, and is not particularly restricted as long as the length of the sequence dose not have an inverse effect on the expression of the fused protein. Here, in the ligation process of the receptor gene with the fluorescent protein gene incorporated in advance in the expression vector, the multi-cloning site held by the expression vector serves as a joint.

As the vector in which a desired promoter or a fluorescent protein gene has been incorporated in advance, those which are commercially available can be used for convenience.

It suffices that the gene coding the hormone receptor protein used in the present invention includes at least the code region which codes the amino acid sequence of the targeted protein, i.e., at least the gene sequence ranging from the initiation codon and the termination codon. That is, the gene coding the hormone receptor protein may include an upstream-side sequence of the initiation codon and a downstream-side sequence of the termination codon, of any length, unless these upstream/downstream-side sequences disturbs the expression of the hormone receptor protein.

For example, in the case of human estrogen receptor β gene (the cDNA sequence thereof and the amino acid sequence thereof are shown in SEQ ID No: 1 and SEQ ID No: 2, respectively), it suffices that the gene includes at least a base sequence of 99 to 1688 position as the coding region. The fusion gene actually produced in the present invention includes a sequence of 53 to 1735 position of the hERβ gene, as disclosed in the examples described below. However, as the length of the sequence of the hERβ gene in the fusion gene varies in accordance with the cloning conditions of the hERβ gene and the conditions in which the fusion gene is prepared, the length of the sequence of the hERβ gene should not be limited to that produced in the examples described below.

The gene coding the fluorescent protein, which is fused to the aforementioned hormone receptor gene in the present invention, is not particularly restricted as long as the gene is a gene of a substance which generates detectable fluorescence., Accordingly, a gene coding any fluorescence-generating material such as GFP, YFP, RFP or the like may be used. Further, the gene coding the fluorescent protein may be of any length, as long as the coding region of the gene is included and the targeted fluorescent protein can be expressed.

The in vitro production of the fluorescence-labeled hormone receptor protein by using the expression vector constructed as described above can be carried out by an in vitro expression method using the common in vitro expression system. Regarding the arrangement of the genes in the expression vector, the genes may be arranged either in the order of the promoter, the receptor and the fluorescent protein or in the order of the promoter, the fluorescent protein and the receptor.

The hormone receptor produced as described above can exist in vitro, while maintaining the morphology as observed in a living cell in which the hormone receptor should naturally exist. Such a fluorescence-labeled hormone receptor can be preserved as an expression vector. Accordingly, if the hormone receptor is synthesized from the expression vector when a test is conducted, denaturation of the hormone receptor during the storage can be avoided. Further, when the hormone receptor is produced in vitro as described above, association molecules which can be a factor of association in FCS measuring (e.g., skeletal proteins of a cell and lipids constituting various organs) can be eliminated. The details of FCS will be described below.

The fluorescence-labeled hormone receptor produced as described above is included within the scope of the present invention. In the fluorescence-labeled hormone receptor as a product, one or a several types of amino acids may be deleted, substituted or added in the amino acid sequence thereof, as long as the intrinsic physiological activity and three-dimensional structure of the hormone receptor are maintained. The method of in vitro producing a fluorescence-labeled hormone receptor by using an expression vector as described above is also included into the scope of the present invention. Further, the expression vector as described above is also included into the scope of the present invention.

Further, the fluorescence-labeled hormone receptor produced as described above may be used without being subjected to further purification or may be used or stored after being purified as described below. Such purification can be carried out by any of known purification means applied to proteins.

5. Fluorescence Correlation Spectroscopy

The fluorescence correlation spectroscopy (i.e., FCS) used in the present invention is a technique in which the fluctuation movement, in the medium, of the targeted fluorescence-labeled molecules is measured and the micro-movements of the individual targeted molecules are accurately measured by using an autocorrelation function (Reference: D. Magde and E. Elson, “Fluorescence correlation spectroscopy. II. An experimental realization”, Biopolymers 1974 13 (1) 29-61).

The FCS is conducted, in the present invention, by: observing Brownian movement of the fluorescent molecules in the solution, in a micro field, by using a laser confocal microscope; analyzing the diffusion time from fluctuation of the fluorescence intensity; and measuring the physical amount (the number, the size of the molecules). The analysis in which molecular fluctuation is detected by FCS in such a micro field as described above is effective in terms of detecting specifically the intermolecular interaction, with high sensitivity.

The principle of detection by FCS conducted in the present invention is described further in detail hereinafter. In FCS, fluorescent signals generated from the micro field of view of the sample are detected and quantitative analyzed by a microscope. At this stage, the targeted, fluorescent-labeled molecules in the medium are constantly moving (i.e., Brownian movement). Accordingly, the fluorescence intensity detected by the microscope changes in accordance with the frequency at which the targeted molecules intrude into the micro field of view and the time in which the targeted molecules stay within the field. For example, if dimerization occurs and the apparent molecular weight is increased, the movement of the targeted molecules is slowed and the apparent number of the molecules decreases. As a result, the frequency at which the targeted molecules intrude into the micro field of view is decreased and the observed fluorescence intensity is changed. By monitoring such changes in fluorescent intensity, the change in the apparent molecular weight of the targeted molecules can be traced.

In the present invention, Fluorescence Intensity Distribution Analysis (FIDA), which allows minute analysis of interactions between molecules of the protein or the like, can be employed in place of FCS (P. Kask, et al., PNAS 23, 96, 13761, 1999; WO98/16814). FIDA is a technique in which fluctuation movement, in a medium, of the fluorescence-labeled targeted molecules is measured, in a micro confocal field (a measurement field) in the order of f (10⁻¹⁵) L excitation-irradiated by laser irradiation, and fluorescence intensity (brightness) per one molecule and the number of the fluorescent molecules are calculated on the basis of the analysis using Poisson distribution function.

Alternatively, Fluorescence Intensity Multiple Distribution Analysis (FIMDA) in which the analysis by FCS and FIDA can be carried out at the same time may be used (K. Palo, Biophysical Journal, 79, 2858-2866, 2000). In FIMDA, data of translational diffusion time of the fluorescent molecules, the number of the molecules and the fluorescence intensity (brightness) per one molecule can be obtained at the same time.

Each of the analysis by FCS, FIDA and FIMDA as described is related to the technique in which the fluctuation movement of fluorescent molecules within a confocal field is measured and the data obtained by the measurement is analyzed by the corresponding function.

6. Device for Fluorescence Correlation Spectroscopy

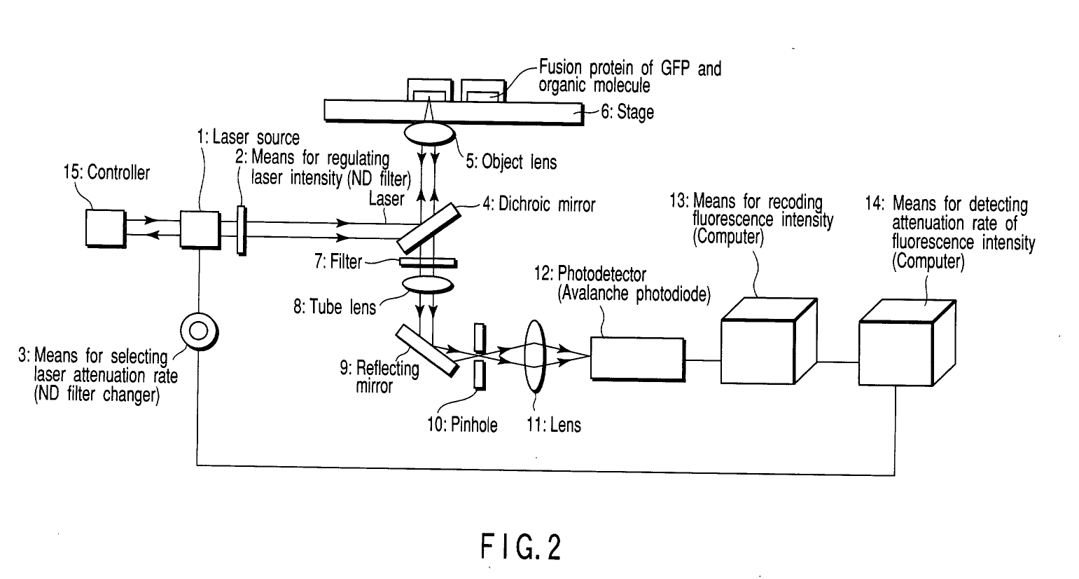

Hereinafter, one example of a device for fluorescence correlation spectroscopy which can be used in the method of the present invention will be described with reference to FIG. 2. As shown in FIG. 2, a device for fluorescence correlation spectroscopy comprises: a laser source 1; means for regulating laser intensity 2 (an ND filter in the present case) for attenuating intensity of light-beam from the laser source 1; means for selecting laser attenuation rate 3 (an ND filter changer in the present case) for setting the laser attenuation rate of the means for regulating laser intensity 2 at an appropriate level; an optical system 4 and 5 for focusing light-beam from the laser source 1 on the sample and forming a confocal field; a stage 6 on which the sample containing the fluorescent molecules is placed; an optical system 7 to 11 for focusing fluorescence emitted from the sample; Photodetector 12 for detecting the focused fluorescence; and means for recording fluorescence intensity for recording changes in fluorescent intensity. As described above, a device for fluorescence correlation spectroscopy uses a confocal laser microscope. In FIG. 2, laser emitted from the laser source 1 may be any of the following lasers: argon laser, helium-neon laser, krypton, helium-cadmium and the like.

In FIG. 2, an optical system 4 and 5 for focusing light-beam from the laser source 1 on the sample and forming a confocal field specifically means a dichroic mirror 4 and an object lens 5. Light-beam emitted from the laser source 1 proceeds along a path as shown in the arrow in FIG. 2. More specifically, the light-beam first has the intensity thereof attenuated in accordance with the arranged degree of attenuation set at the means for regulating laser intensity 2 (an ND filter in the present case); refracted by the dichroic mirror 4 toward the stage at the angle of 90° with respect to the incident light; and irradiated on the sample on the stage 6 by way of the object lens 5. The light-beam is focused on the sample at one micro point in such a manner, whereby a confocal field is formed.

In the present invention, the sample placed on the stage 6 may be either a solution in which the fluorescent molecules are suspended or a biomolecule such as a protein labeled with the fluorescent molecules. The fluorescent molecules can be produced by a method in which a fused protein of the fluorescent protein (e.g., green fluorescent protein) and the targeted protein to be analyzed is expressed by using the known genetic engineering technique.

In FIG. 2, fluorescence emitted from the fluorescent molecules in the confocal field is focused by the optical system 7 to 11. More specifically, the fluorescence proceeds through a filter 7 and a tube lens 8, is refracted by a reflecting mirror 9, forms an image at a pin hole 10, passes through a lens 11 and is focused on the photodetector 12.

The photodetector for detecting the focused fluorescence 12 (an Avalanche photodiode in the present case) converts the received light signals to electric signals and transmits the electric signals to the means for recording fluorescence intensity 13 (a computer in the present case).

The means for recording fluorescence intensity 13 for recording changes in fluorescence intensity carries out recording and analysis of the data on fluorescence intensity which has been transferred thereto. Specifically, the means for recording fluorescence intensity 13 sets an autocorrelation function on the basis of the analysis of the fluorescence intensity data. An increase of molecular weight and a decrease in the number of molecules due to the movement of the fluorescent molecules (e.g., dimerization of the fluorescent molecules) or a decrease in the number of molecules due to binding of the fluorescent molecules to a specific DNA region can be detected on the basis of changes in the autocorrelation function.

A device for carrying out the aforementioned FCS is also included into the scope of the present invention.

7. Method of detecting presence/absence of action of test substance, on hormone receptor

Presence/absence of an action of the test substance, on the hormone receptor, can be detected by: reacting the fluorescence-labeled hormone receptor synthesized as described above with the test substance under an appropriate condition; measuring molecular fluctuation by FCS, with fluorescent intensity being used as an index; and setting the autocorrelation function on the basis of the measured data. More specifically, presence/absence of an action of the test substance, on the hormone receptor, may be detected by comparing the data prior to the reaction with the data after the reaction. Or, presence/absence of an action of the test substance, on the hormone receptor, can be detected by comparing the data obtained in the presence of the test substance with the data obtained in the absence of the test substance.

Regarding the predetermined or appropriate conditions required for detecting an action of the test substance, on the fluorescence-labeled hormone receptor, e.g., the reaction temperature, the reaction time, and the composition of the reaction solution, the researcher conducting the test can select any suitable conditions, in accordance with the types of the fluorescence-labeled hormone receptor and the test substance used in the test.

When a test substance is added to a targeted, fluorescent-labeled hormone receptor and the mixture is maintained in the pre-set appropriate condition according to the present invention, if the test substance has an action on the hormone receptor, the substance is bound to the hormone receptor. As a result, a series of biochemical reactions occur as in the case with the “true” ligand and the state of the receptor is changed, whereby the fluorescence intensity detected by FCS changes. By using such a change as an index, whether or not the test substance has an action on the hormone receptor is determined. In a case where there is observed no change in fluorescent intensity between prior and after the addition of the test substance, it is determined that the test substance has no action on the hormone receptor.

In the foregoing description, a method of detecting presence/absence of an action of the test substance, on the fluorescence-labeled hormone receptor, has been described as one embodiment of the present invention. However, the present invention is not restricted to an embodiment using a hormone receptor. That is, in the present invention, a protein such as antigen, antibody or the like may be produced by using an expression vector (here, the protein can be produced as a fused protein of such antigen or the like and a fluorescence-labeled material) and then a substance which is specifically bound to the protein may be detected by using the FCS technique.

(Advantageous Effect of First Embodiment)

According to the present invention, a method of accurately detecting presence/absence of binding capacity, of a test substance, with respect to a protein, is provided.

Specifically, according to the present invention, it is possible to synthesize, in vitro, a protein maintaining the three-dimensional structure which should naturally be observed in vivo (that is, this protein is a biomolecule which is intrinsically produced in vivo. The present invention enables in vitro production of such a biomolecule, with maintaining the inherent three-dimensional structure thereof). Further, according to the present invention, it is possible to provide the protein with fluorescent labeling, while maintaining such an intrinsic three-dimensional structure. As a result, it is possible to simulate the in vivo behavior of the targeted protein, in the cell-free condition (i.e., in vitro). In short, by reacting a test substance with the targeted protein in such a state as described above, it is possible to test the affinity of the test substance with respect to the targeted protein. Further, by conducting tests in such a condition as described above, association with irrelevant molecules (such as the skeletal proteins of cells and lipids constituting various organs) in a cell, which association is a problem in the conventional method using FCS, can be avoided. Therefore, according to the present invention, it is possible to obtain significantly accurate test results, as compared with the conventional method. Further, in the present invention, it is possible to quantitatively assay the affinity of the test substance with respect to the targeted protein.

The fusion gene constituted of GFP and a biomolecule, which is produced according to the present invention, can be stored in the stable manner. Accordingly, the fusion protein of the present invention can be stored as the fusion gene for a long period in the stable manner, without either experiencing changes in the three-dimensional structure or losing functional activity thereof (on the contrary, change in the three-dimensional structure and loss of functional activity of the protein during long-term storage is inevitable in the conventional method). Further, according to the present invention, as the measurement of behavior of the fusion protein by FCS can be carried out immediately after the fusion gene thereof is transcriptionally activated and the protein is expressed in vitro, occurrence of change in the three-dimensional structure and resulting loss of functional activity of the protein, during the reaction, can be suppressed minimum.

By producing a fluorescence-labeled hormone receptor according to the present invention and detecting presence/absence of binding capacity, of a test substance, with respect to the obtained hormone receptor, it is possible to screen presence/absence of an endocrine disrupting action of the test substance in the simple and easy manner.

The estrogen receptor, in particular, is localized in the nucleus. In the conventional method of detecting a fluorescence-labeled hormone receptor in a cell by using FCS, it is difficult to measure the fluctuation of molecules localized in the nucleus. On the contrary, according to the present invention, it is possible to conduct accurate and easy measuring, by FCS, of the behavior of the fluorescence-labeled hormone receptor molecules. The chemical materials which are bound to the estrogen receptor are the source of environmental pollution, as one group of endocrine disrupting chemicals. The present invention can be utilized as one method of screening such endocrine disrupting chemicals.

<Second Embodiment>

The content studied in the process of completing a second embodiment of the present invention will be described in detail hereinafter.

When detection is carried out during the process of dimerization of the receptor, it is difficult to detect or distinguish the receptor monomer from the receptor dimer on the basis of the difference in the diffusion time, because the diffusion constant of the receptor monomer is less than twice as much as that of the receptor dimer. On the other hand, the diffusion constant of the receptor monomer can be made at least twice as much as that of the receptor/ligand/DNA complex, depending on the size of DNA. If the diffusion constant of the receptor monomer can be made at least twice as much as that of the dimer/complex of the receptor by setting the size of DNA at an adequate level, the binding reaction of the receptor is likely to be detected with clear distinction between the receptor monomer and the dimer/complex of the receptor.

It is generally assumed from the discoveries in the past that, in order to detect or distinguish molecules of two types which are different in molecular size, with clear distinction in results between the two types, it is critical that the diffusion constant of one type of the molecule is at least two times as much as larger/smaller than the diffusion constant of the other type.

Here, when it is assumed that a molecule is spherical and the radius thereof is r, the diffusion constant D of the molecule is defined by the following formula, by using the radius r, according to the Einstein-Stokes formula:

D=(κ_{B}T)/(6πηr) (A)

wherein κ_{B} is Boltzman's constant;

T is the absolute temperature; and

η is the viscosity of the solvent solution.

It is generally assumed that the receptor proteins are spherical molecules and the diffusion constant thereof changes according to the aforementioned formula (A).

On the other hand, it is assumed that the DNA fragment having the targeted DNA sequence is a rod-shaped molecule and the diffusion constant D of the molecule is defined by the following formula (B):

D=(AκT)/(3πη₀L) (B)

wherein A=In(L/d)+0.312+0.565/(L/d)−0.1 (L/d)²;

L is the length of DNA (3.4 Å× the number of base pairs [bp]);

d is the diameter of the rod-shaped molecule of DNA (23.8 Å);

κ is Boltzman's constant;

T is the absolute temperature; and

η₀ is the viscosity of the solvent solution.

The diffusion time of the molecule (τ_{diff}) obtained by the FCS measurement is defined by the following formula.

τ_{diff}=ω²/4D

wherein ω represents the diameter of laser beam irradiated in the FCS measurement.

When a receptor protein molecule is compared with a rod-shaped DNA molecule having approximately the same molecular weight as the receptor protein molecule, the diffusion constant of the DNA molecule as a rod-shaped molecule is generally smaller than that of the receptor protein as a spherical molecule (in other words, the diffusion time of the DNA molecule is longer than that of the receptor protein). For example, estrogen receptor β, which is one of the nuclear receptors, is presumably a spherical molecule whose molecular weight is approximately 60 kDa, and the calculated diffusion constant thereof is approximately 7.5×10⁻¹¹ (m²/S). If it is assumed that the receptor is bound to a ligand and a dimer is formed, the diffusion constant of the dimer is, according to the calculation, 5.9×10⁻¹¹ (m²/S). Accordingly, in this case, the diffusion constant of the receptor monomer is less than twice as much as the diffusion constant of the dimer, whereby it is concluded that detecting or distinguishing the dimer from the receptor monomer by the difference in diffusion time will be difficult.

On the contrary, a double stranded DNA whose molecular weight is approximately 60 kDa has a length of 100 bp or so, and the diffusion constant thereof is 3.8×10⁻¹¹ (m²/S), which is approximately the half of the diffusion constant of a spherical molecule having the same molecular weight (i.e., approximately 60 kDa) as that of this DNA. Accordingly, if the receptor/ligand complex is bound to a DNA fragment of an appropriate length and forms a larger complex, the diffusion constant of the resulting complex will possibly be at least twice as small as the diffusion constant of the receptor monomer.

As a result, by detecting presence/absence of the receptor/ligand/DNA fragment complex from the measurement result of the diffusion time, it is possible to clearly detect presence/absence of the binding reaction of the test substance to the receptor. The inventors of the present invention have newly discovered this feature and completed the present invention.

Hereinafter, the receptor, the test substance and the DNA fragment having a specific DNA sequence, which can be employed in the second embodiment, will be described in detail.

<Receptor>

The receptor used in the present invention is not particularly restricted, as long as the receptor receives a ligand, is bound to the specific DNA sequence in the nucleus and causes an action thereon, in vivo.

Examples of such a receptor include a receptor belonging to the nuclear hormone receptor superfamily, which is bound to a specific DNA sequence and functions as a transcription-regulating factor (which receptor will be also referred to as a “nuclear receptor”). Specific examples of the nuclear receptor include: a receptor whose intrinsic or “true” ligand has been identified, such as the estrogen receptor, the progesterone receptor, the thyroid hormone receptor, and the glucocorticoid receptor; and a receptor whose intrinsic or “true” ligand has not been identified (an orphan receptor, for example). Among the aforementioned examples, the estrogen receptor, for which a large-scale, high-throughput screening of substances deemed as potential ligands are being conducted, is especially important.

In the present invention, “the marker material which can generate a light signal” for labeling the receptor molecule is not particularly restricted, as long as the marker material is capable of generating a detectable light signal. A material which emits fluorescence or a material which effects chemical luminescence can be used, for example. As “the marker material which can generate a light signal”, any fluorescent material which emits fluorescence can be preferably used. Among such fluorescent materials, a fluorescent protein which emits light without requiring addition of any substrate is especially preferable. Examples of the fluorescent protein include GFP (Green Fluorescent Protein), CFP (Cyan Fluorescent Protein), YFP (Yellow Fluorescent Protein) and RFP (Red Fluorescent Protein) and the like. Examples of the method of labeling a receptor molecule with a fluorescent material include a method based on chemical modification by a chemical reaction and a method based on genetic engineering. In particular, when the gene of the targeted receptor is known, a fusion gene coding a fused protein of the fluorescent protein (such as GFP) and the receptor can be prepared by genetic engineering and then the fused protein can be produced by using the obtained gene, according to a method such as in vitro translation. As such a method allows production of a fluorescence-labeled receptor in the order of mg, the amount which is necessary for conducting screening can be easily prepared. When the fluorescence-labeled receptor has been prepared as described above, it is necessary to confirm, before actually using the receptor, whether or not the fluorescent-labeled receptor maintains the intrinsic functions thereof as a receptor.

Specifically, regarding the example in which a fused protein of estrogen receptor a and a fluorescent protein GFP (green fluorescent protein) is produced according to a genetic engineering method, please refer to Han Htun, Laurel T. Holth, Dawn Walker, James R. Davie, and Gordon L. Hager (1999) Direct Visualization of the Human Estrogen Receptor a Reveals a Role for Ligand in the Nuclear Distribution of the Receptor., Mol. Biol. Cell, 10, 471-486.

By using a receptor molecule which has been fluorescence-labeled as described above, use of the labeled ligand of a known type is rendered unnecessary (use of the labeled ligand of a known type is necessary in a receptor-binding assay of the conventional type). Therefore, the receptor to be analyzed in the present invention is not restricted to a receptor whose “true” ligand has been identified. Further, the process of labeling a test substance with fluorescence conducted in the conventional method, which process is laborious and time/cost-consuming, can also be rendered unnecessary.

<Test Substance>

Examples of “a test substance” used in the present invention include any chemical substances suspected of having an endocrine disrupting action on the aforementioned receptor.

<Fragment of Nucleic Acid Containing Specific Nucleic Acid Sequence>

In the present invention, the specific nucleic acid sequence to which a receptor/ligand complex can bind itself is not particularly restricted, as long as the receptor which has received the ligand can identify the specific nucleic sequence and bind itself thereto.

In the present specification, “a nucleic acid” may generally be formed by nucleotide which constitutes DNA or RNA (which nucleotide will be also referred to as “simple nucleotide” hereinafter) or may include modified nucleotide (a phosphate ester of inosine, methyladenosine, methylguanosine or the like). In the present invention, “a nucleic acid” is preferably formed by nucleotide which constitutes DNA. In the description hereinafter, a nucleic acid will be regarded as DNA for convenience.

The motif of base sequence to which a nuclear receptor can generally be bound has already been analyzed. The typical sequence motif includes 15 bp and has a structure in which inverted palindrome of 6 bp interposes spacer sequence of 3 bp. For example, the specific DNA sequence to which the estrogen receptor having received a ligand can be bound is 5′-AGGTCANNNTGACCT-3′ (N represents any nucleotide which is a simple nucleotide; SEQ ID No: 3). A specific DNA sequence to which a receptor can be bound may be designed and used in an appropriate manner, in accordance with the type of the receptor.

In the present invention, the DNA fragment having the aforementioned specific DNA sequence must include a specific DNA sequence to which the receptor/ligand complex can be bound. Additionally, the DNA fragment must have the appropriate length. Here, “the appropriate length” represents a length necessary for clearly distinguishing the receptor monomer from the receptor/test substance/DNA fragment complex in the detection process, on the basis of the difference in diffusion time. More specifically, “the appropriate length” represents a length which allows to make, the diffusion constant of the receptor/test substance/DNA fragment complex, at least twice as small as the diffusion constant of the receptor monomer.

The appropriate length of a DNA fragment is preferably set as described below.

It is preferable that the DNA fragment having the aforementioned specific DNA sequence is designed so as to have a molecular weight which is no smaller than that of the receptor. Alternatively, it is preferable that the DNA fragment having the aforementioned specific DNA sequence is designed so as to have the diffusion constant which is no larger than that of the receptor.

For example, in the case of estrogen receptor β (a spherical molecular whose molecular weight is approximately 60 kDa), a DNA fragment having a molecular weight which is approximately equal to that of the receptor can be obtained by double stranded DNA of approximately 100 bp. Accordingly, in this case, an appropriate DNA fragment is designed so as to have length of approximately 100 bp or more. Further, the diffusion constant of estrogen receptor β is approximately 7.5×10⁻¹¹ (m²/S) according to calculation and a DNA fragment having diffusion constant approximately equal to that of the receptor is obtained by double stranded DNA of approximately 30 bp. Accordingly, in this case, an appropriate DNA fragment is designed so as to have length of approximately 30 bp or more. Although there is generally no upper limit in the length of the DNA fragment, it is suggested in the present invention that the length of the DNA fragment does not exceed several kb or so.

More specifically, for the DNA fragment having a specific DNA sequence, double stranded DNA whose length is preferably 100 to 4500 bp or more preferably 100 to 200 bp can be used. Single stranded DNA is not preferable because single stranded DNA forms base pairs within the molecule of its own and tends to have a three-dimensional structure.

In the case where estrogen receptor β is used, double stranded DNA fragment, which includes the estrogen-responsive sequence {5′-AGGTCANNNTGACCT-3′ (N represents any nucleotide which is a simple nucleotide; SEQ ID No: 3)} as a specific DNA sequence, as well as other optional sequences, and whose length is 100 to 200 bp, is preferably used.

If the DNA fragment has the predetermined length as described above and includes a specific DNA sequence to which the receptor/ligand complex can be bound, the base sequences at other portions thereof are not restricted and are optional. In other words, in the DNA fragment, the base sequences other than the aforementioned specific DNA sequence do not have so much meaning, as long as the length of the DNA fragment is set at the appropriate length which allows clear distinction between the receptor monomer and the receptor/DNA complex during the detection process.

<Process of Maintaining Reaction Components in Predetermined Solution>

Next, the process of maintaining the aforementioned receptor, the test substance and the DNA fragment including the specific DNA sequence in a predetermined solution will be described.

The predetermined solution is not particularly restricted, as long as the receptor and the ligand can form a complex and the receptor/ligand complex can bind itself to the specific DNA sequence, which specific DNA sequence allows the complex to be bound thereto. As the predetermined solution, a buffer which is used for the DNA-protein binding reaction in a gel-shift assay can generally be employed. For example, as the predetermined solution, 20 mM Tris-HCl (pH 7.9), 1 mM DTT, 1 mM EDTA (pH 8.0), 12.5% glycerol, 0.1% Triton X-100, 50 μg/mL poly (dI-dC), 250 μg/mL BSA, 50 to 100 mM KCl can be used.

The receptor, the test substance and the specific DNA fragment are maintained in the predetermined solution at an appropriate concentration. In general, the purified receptor protein, the test substance, and the DNA fragment having the specific DNA sequence are maintained in the predetermined solution at the concentrations of 0.03 to 5 μg/mL, 10⁻¹² to 10⁻⁶ M, 50 to 500 nM, respectively. The conditions during the binding reaction (temperature, pH, time) may be appropriately set, depending on the type of the receptor protein. The process of maintaining the reaction components in the solution is preferably carried out by incubating the solution for a predetermined period.

An example of the binding reaction includes the steps of: adding a purified receptor protein and a test substance, to a solution containing 20 mM Tris-HCl (pH 7.9), 1 mM DTT, 1 mM EDTA (pH 8.0), 12.5% glycerol, 0.1% Triton X-100, 50 μg/mL poly (dI-dC), 250 μg/mL BSA, and 100 mM KCl, such that the concentrations of the receptor protein and the test substance are 0.03 to 5 μg/mL and 10⁻¹² to 10⁻⁶ M, respectively; incubating the solution at 22° C. for 10 minutes; adding the DNA fragment having the specific DNA sequence to the solution, such that the concentration thereof is 50 to 500 nM; and further incubating the solution for 30 minutes to 1 hour.

For effecting incubation, the reaction solution, i.e., the aforementioned solution containing the set of the reaction components in the suspended state, can be held by an appropriate liquid holding means such as a test tube, a well, a cuvette, a groove, a pipe, a plate, and a porous material. Here, it is preferable that the shape, material, size and the like of the liquid holding means are selected so that a part or all of the various detection steps, including distribution of the solution to plural containers, stirring, incubation, measurement, transfer of the solution, can be swiftly carried out. For example, in the case of the measurement by FSC, as the measurement is carried out in a extremely micro field of the optical focus level, a liquid holding means of a very small size may be employed. When detection is effected by optical measurement, in particular, it is preferable that the liquid holding means has an opening which allows entry and/or exit of light-beam for measurement, so that the light-beam for measurement has a direct action on the reaction components.

By maintaining the aforementioned receptor, the test substance and the DNA fragment having the specific DNA sequence in the predetermined solution, the following reactions occur.

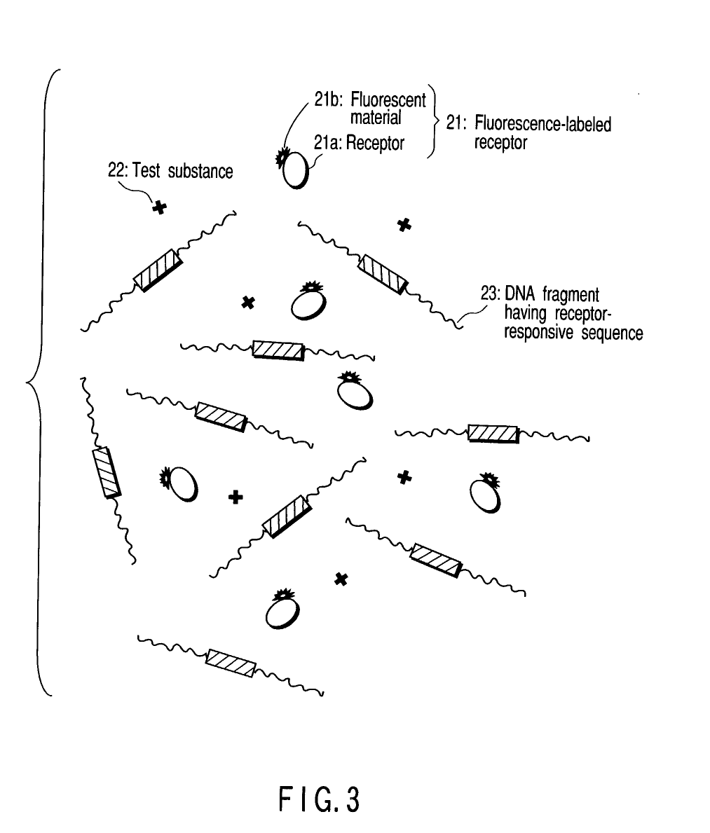

FIG. 3 is a view of a reaction solution model in which the test substance is not capable of being the ligand of the specific receptor. In FIG. 3, the receptor 1a has been labeled with a fluorescent material 1b, so that the receptor la can be traced. The DNA fragment 3 having a receptor-responsive sequence represents the DNA fragment having a sequence to which the complex of the receptor and the ligand can be specifically bound. Here, “a reaction solution” represents a liquid which is the predetermined solution containing the set of the reaction components suspended therein.

In the case shown in FIG. 3, as the test substance 2 cannot be the ligand of the fluorescence-labeled receptor 1, the test substance 2 cannot be bound to the fluorescence-labeled receptor 1 contained in the reaction solution, thereby is not reacted with the DNA fragment 3 having the receptor-responsive sequence. Therefore, the complex of the receptor, the test substance and the DNA fragment is not formed after the incubation for a predetermined period, and the receptor, the test substance and the DNA fragment each exist in the separately suspended state.

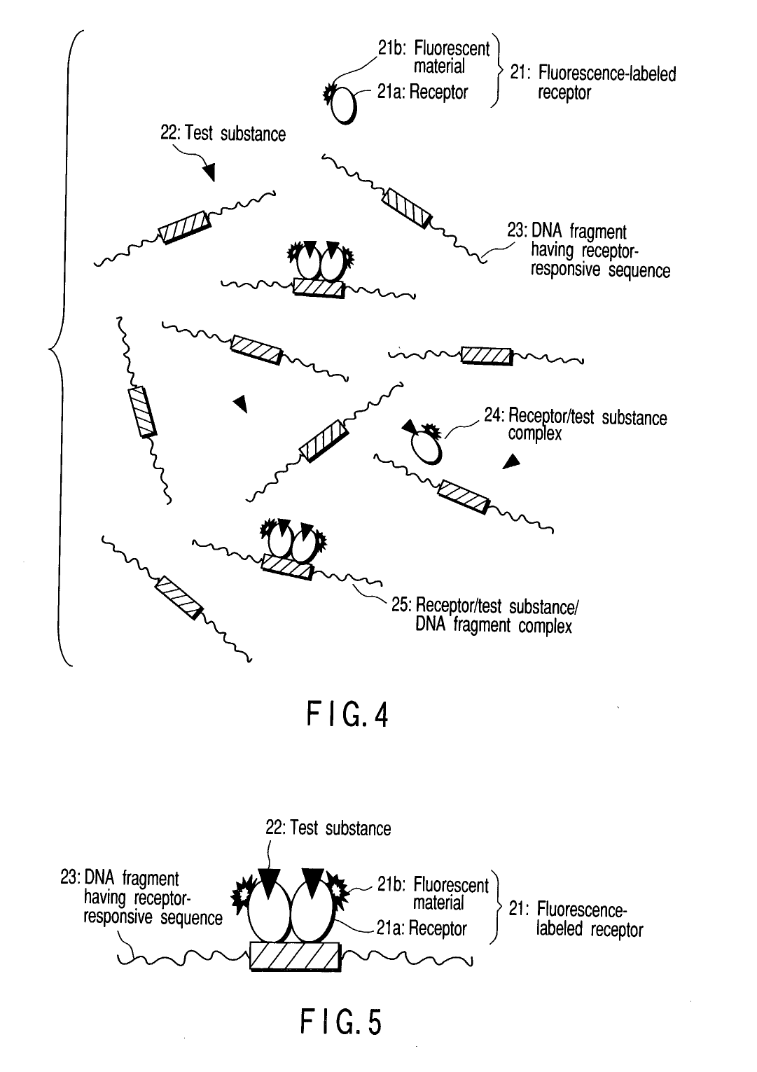

FIG. 4 is a view of a reaction solution model in which the test substance is capable of being the ligand of the specific receptor. The receptor 1a has been labeled with a fluorescent material 1b so that the receptor 1a can be traced, as in FIG. 3. The DNA fragment 3 having a receptor-responsive sequence represents the DNA fragment having a sequence to which the complex of the receptor and the ligand can be specifically bound.

In the case of FIG. 4, during the incubation process for a predetermined period, the test substance 2 is bound to the fluorescence-labeled receptor 1 in the reaction solution and forms the complex 4 of the receptor and the test substance. The complex further forms a dimer and then is bound to a DNA fragment 3 having the receptor-responsive sequence, thereby forming the complex 5 of the receptor, the test substance and the DNA fragment.

FIG. 5 shows the structure of the receptor/test substance/DNA fragment complex 5 formed as described above. Specifically, FIG. 5 shows the complex of the receptor dimer constituted of two fluorescence-labeled receptors and the DNA fragment having the receptor-responsive sequence. It should be noted that, although the receptor/test substance complex forms a dimer and then is bound to the DNA fragment in FIG. 4, there may also exist a receptor/test substance complex which does not form a dimer with another complex and is bound, as a monomer, to the DNA fragment.

<Process of Detecting Presence/Absence of Receptor/Test Substance/DNA Fragment Complex>

The receptor, the test substance, and the DNA fragment having the receptor-responsive sequence are maintained in the predetermined solution, as described above. Thereafter, presence/absence of the receptor/test substance/DNA fragment complex is detected. This detection is preferably carried out by measuring the diffusion time of the fluorescence-labeled receptor by a suitable means such as FCS. As a result, whether or not the test substance can be a ligand of the targeted receptor can be detected or determined.

The specific procedure of measurement by using FCS will be described below. However, the present invention is not restricted to the following measuring method.

First, the targeted receptor is labeled with fluorescence and the diffusion time of the receptor as a monomer in the reaction solution is measured by FCS. Next, the test substance and the DNA fragment having the receptor-responsive sequence are added to the reaction solution containing the receptor, and the mixture is incubated for a predetermined period so that the binding reaction proceeds. Thereafter, the diffusion time of the fluorescence-labeled receptor in the reaction solution is measured by FCS, as is done for the receptor as a monomer.

Here, in the case where the test substance cannot be the ligand of the receptor, the receptor exists as a monomer in the reaction solution after the incubation, without forming a dimer or a complex, whereby the diffusion time thereof does not change between before and after the incubation (refer to FIG. 3).

On the contrary, in the case where the test substance can be the ligand of the receptor, the complex of the receptor, the test substance and the DNA fragment is formed after the incubation. When such a complex is formed, the apparent molecular weight of the labeled receptor increases and thus the diffusion time of the receptor increases, as compared with the diffusion time of the receptor as a monomer (refer to FIG. 4). However, there also exist the receptor molecule to which the test substance has not been bound and remains as a monomer, in the reaction solution.

Accordingly, if the diffusion time of the fluorescence-labeled receptor monomer is set as a fixed value and an autocorrelation function is set, formation of the receptor-the test substance-the DNA fragment complex can be detected. Further, for each of the receptor monomer and the complex in the reaction solution, the diffusion time and the proportion thereof occupied in the group of the fluorescent molecules as a whole can be calculated, respectively. As a result, it is possible to detect the degree at which the receptor/test substance/DNA fragment complex has been formed and also evaluate the binding affinity of the receptor/test substance complex with respect to the targeted DNA. Time of a few to tens of seconds suffices as the time to be spent for measuring the diffusion time. The amount of the reaction solution required for the measurement does not exceed tens of microliter (μl). Therefore, by employing a detection system using FCS, substances of a variety of types can be rapidly assayed in a highly sensitive manner, although the amounts of the substances are very small.

(Effect of Second Embodiment)

As described above, the method of detecting the binding reaction of the receptor and the test substance, of the present invention, comprises the steps of: adding the DNA fragment having the targeted DNA sequence to which sequence the receptor is specifically bound, to the detection system; and detecting whether or not the receptor/testes substance/DNA fragment complex is formed. Therefore, according to the detection system of the present invention, to which a DNA fragment is added, both the receptor as a monomer and the receptor/testes substance/DNA fragment complex can be detected with sufficiently clear distinction therebetween and thus the binding reaction of the receptor and the test substance can be detected with clear distinction between reaction-positive and reaction-negative.

Further, according to the method of the present invention, it is possible to detect degree at which the receptor/test substance/DNA fragment complex has been formed, in a form of a relative value. Thus, on the basis of the obtained relative values which indicate the degree of the complex formation, the binding affinity of the ligand/receptor complex with respect to the targeted nucleic acid can also be evaluated.

The method of the present invention is inherently a detection system in a solution and is suitable for a high-throughput test. Moreover, the present method satisfies the requirements of a high-throughput test, as follows. That is, according to the present invention, it is possible to separately detect the receptor molecule,to which the test substance has not been bound (a monomer) and the receptor molecule which has formed a complex with the DNA fragment, without isolating these molecules from the reaction solution. Therefore, the separation/washing process of the molecules, which is generally quite troublesome or complicated, is not required. Further, neither addition of a labeled ligand of the known type nor fluorescence-labeling all the substances to be tested is required. Yet further, time of a few to tens of seconds suffices as the time to be spent for measuring the diffusion time and the amount of the reaction solution required for the test does not exceed tens of microliter (μl). Therefore, test substances of a variety of types can be rapidly assayed in a highly sensitive manner, although the amounts of the substances are very small.

As described above, according to the detection system of the present invention, to which the DNA fragment is added, the binding reaction of the receptor-test substance complex, with respect to the targeted DNA sequence, which reaction is presumably correlated with the activity as a ligand of the test substance, can rapidly detected in the highly sensitive manner.

Further, if the target DNA is unknown for a receptor to be used, the present invention can effectively be applied for screening the target DNA sequence of the receptor, by adding DNA fragments having various sequences to the binding reaction solution.

### EXAMPLES

### 1. Production of Recombinant Protein for Confirming Function of Fusion Protein of GFP+Estrogen Receptor β

<Object of Experiment>

The object of the present example is: to confirm, by the in vitro expression method, that the targeted protein is expressed from the cloned (GFP+estrogen receptor (ER) β) gene; to confirm, in vitro, that the (GFP+estrogen receptor β) protein forms a dimer; and to check the function of the (GFP+estrogen receptor β) protein.

<Method of Experiment>

By using a transcription/translation system of a vector for in vitro expression (TNT Quick Coupled Transcription/Translation System, manufactured by Promega co.) containing the cloned (GFP+estrogen receptor β) gene and a transcription/translation system of a vector for in vitro expression (the same type) containing the “estrogen receptor β only” gene, an expression experiment of the gene product of the cloned (GFP+estrogen receptor β) gene and an expression experiment of the gene product of the “estrogen receptor β only” gene are performed. The expression and antigenicity are confirmed by the western blotting method. The dimer-forming function is also confirmed.

<Experiment Procedure and the Results>

1) Construction of vector for in vitro expression

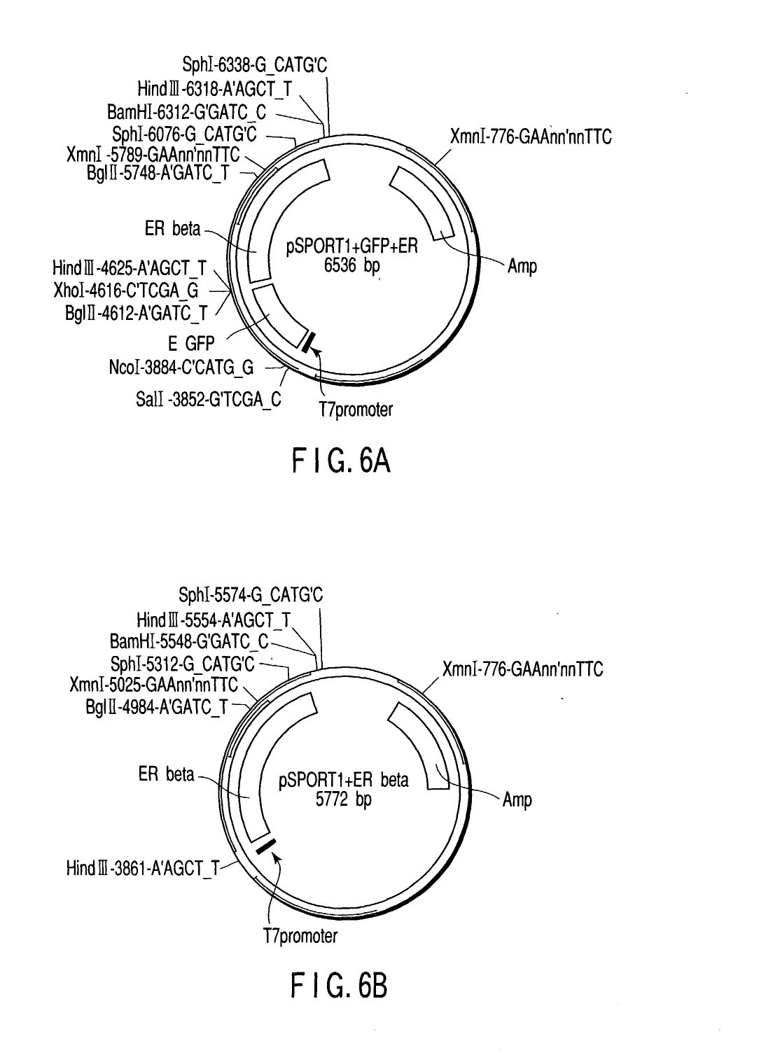

As a vector for expressing the protein by using an in vitro transcription/translation system, pSPORT1 (manufactured by Lifetech Oriental co., plasmid pSPOR1) was selected. This vector includes the sequence of T7RNA promoter required for RNA synthesis, as well as the restriction enzyme sites (SalI, BamHI) located at the downstream side of the promoter sequence, at which sites the fusion gene of (GFP+ERβ) can be introduced. Each of the fusion gene of (GFP+ERβ) (refer to FIG. 6A) and the ERβ gene (refer to FIG. 6B), each of which had been cut out from the vector constructed for cell expression and purified by electrophoresis, was incorporated at the restriction enzyme sites (refer to FIG. 6).

A plasmid (pEGFP+ERβ), which contained the fusion of GFP gene and the ERβ gene (i.e., the vector for cell expression), was prepared and isolated, and digested by using the restriction enzymes.

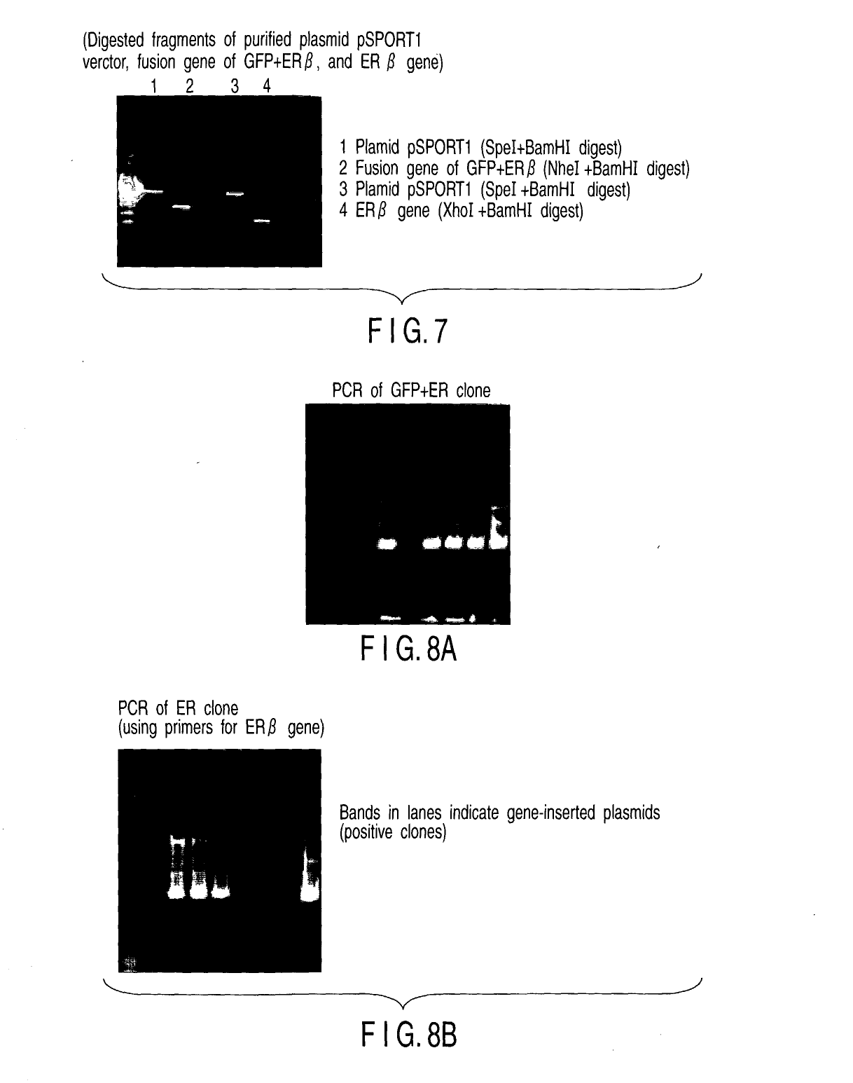

The digested plasmid was confirmed by electrophoresis. After a large quantity of the digested plasmid was subjected to electrophoresis, the fragments of the fusion gene of (GFP+ERβ) and the ERβ gene were cut out by the gel-cutting out method, and the fragments were purified from gel by the DNA extraction method using glass beads (manufactured by Bio101 co., Geneclean III). After the purification process, the concentration and purity were checked by electrophoresis. In a manner similar to the plasmid (pEGFP+ERβ), the plasmid pSPORT1 vector having T7 promoter sequence was digested by the restriction enzyme and then purified by the DNA extraction method using glass beads. After the purification process, the concentration was checked by electrophoresis (FIG. 7).

The digested pSPORT1 vector and the fusion gene of (GFP+ERβ) were mixed. The digested pSPORT1 vector and the ERβ gene were mixed. Each set of the gene mixtures was subjected to ligation, transformed to Escherichia coli DH5α and cultured on a plate containing ampicillin (i.e., an Amp+plate).

A colony was selected for each set and transferred to a PCR tube. Thereafter, PCR was carried out by using the primer for ERβ. Amplification was confirmed by electrophoresis.

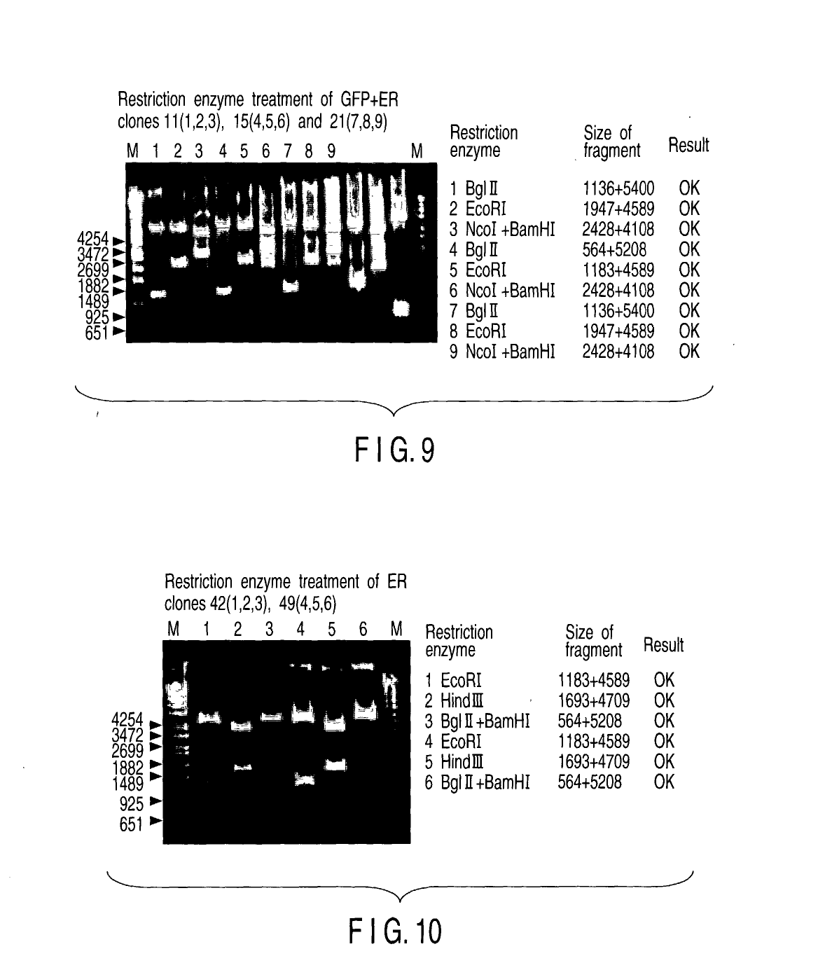

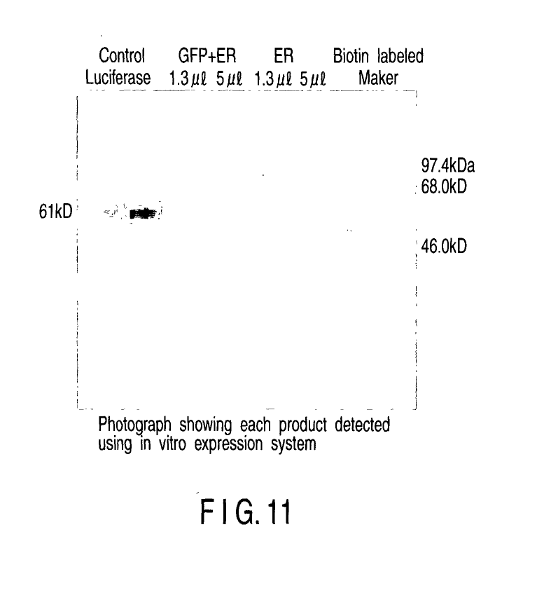

No less than 4 clones of recombinants were obtained in the set of the pSPORT1 vector and the fusion gene (GFP+ERβ). No less than 3 clones of recombinants were obtained in the set of the pSPORT1 vector and the ERβ gene (FIG. 8). The plasmids were isolated from these clones and digested by using several types of the restriction enzyme. It was then confirmed that the obtained plasmid was a correct recombinant (FIG. 9 and FIG. 10). As a result of the operations as described above, a plasmid containing the fusion gene of (GFP+ERβ) (FIG. 9) and a plasmid containing the ERβ gene (FIG. 10), which were ready for being used for the in vitro expression, were obtained.

2) In vitro expression

The expression of the protein was carried out in the in vitro expression system, by using the expression vectors constructed as described above. For the expression process, TNT Quick Coupled Transcription/Translation System, manufactured by Promega co. was used. By adding tRNA having lysine (one of the amino acids) labeled with biotin, during protein synthesis by expression, the biotin-labeled lysine was incorporated to the protein which had been produced by expression (Transcend Non-Radioactive Translation Detection System, manufactured by Promega co., was used). As the control of the in vitro expression system, the luciferase gene attached to the kit was expressed. The reaction solution after the in vitro expression was subjected to SDS polyacrylamide gel electrophoresis for separation. Detection was carried out on the basis of Bromophenol Blue staining or the color generation reaction on the streptoavidin-alkali phosphatase based membrane after western blotting transfer (attached to Transcend Non-Radioactive Translation Detection System, manufactured by Promega co.).

Result

In the case where the reaction solution of the in vitro expression was subjected to SDS polyacrylamide gel electrophoresis for separation and then Bromophenol Blue staining was conducted, most of the observed bands were those of the proteins contained in the reagents of the in vitro expression system, and the protein bands which should be specifically observed in the samples (i.e., the sample in which the fusion gene of (GFP+ERβ) was expressed and the sample in which the ERβ gene was expressed) were not observed.

In the case where the reaction solution of the in vitro expression was subjected to SDS polyacrylamide gel electrophoresis for separation, followed by western blotting transfer and then detection on the basis of the color generation reaction on the streptoavidin-alkali phosphatase based membrane was carried out, the sample in which the fusion gene of (GFP+ERβ) had been expressed exhibited a band of the expressed protein of approximately 80 kDa, and the sample in which the ERβ gene had been expressed exhibited a band of the expressed protein of approximately 60 kDa. Also, the sample in which the gene of luciferase was expressed exhibited the expected band which corresponded to 61 kDa, proving that the in vitro expression system was certainly functioning (FIG. 11).

3) Purification method by antibody

The protein solution, which had been synthesized in vitro as described above, was collected and purified as follows. First, coarse purification was carried out by ion-exchange chromatography by using DE-52 or CM-52 cellulose. Purification was then carried out by selecting the optimum condition which enabled separation of impure protein. The optimum condition was selected from the following conditions: the gradient-eluting conditions in which the concentration of NaCl was varied from 0.1 to 0.3 M was employed, and the pH of the equilibrium buffer solution was changed from 3.0 to 7.0. It was observed that the estrogen receptor protein was excellently separated from hemoglobin when the pH of the buffer solution was the optimum (peak) value. Further, purification was carried out by affinity chromatography using the monoclonal antibody specific to the estrogen receptor. CN-Br activated Sepharose 4B (Pharmacia) was used as the affinity column, which served as the carrier of affinity chromatography. As the equilibrium buffer solution of the column, 0.2 M PB, 0.2 M NaCl, pH 6.5 was used. 5M guanidine chloride was used for elution.

When the estrogen receptor collected in the aforementioned purification method was analyzed by the fluorescence correlation analyzing method, the date on the estrogen receptor was obtained as one molecule of fluorescent molecule.

4) Endocrine disrupting chemicals-detecting system employing GFP-ER obtained by in vitro transcription

A system of detecting endocrine disrupting chemicals was successfully established by using the aforementioned GFP-ER protein as a sample and adding estradiol thereto.

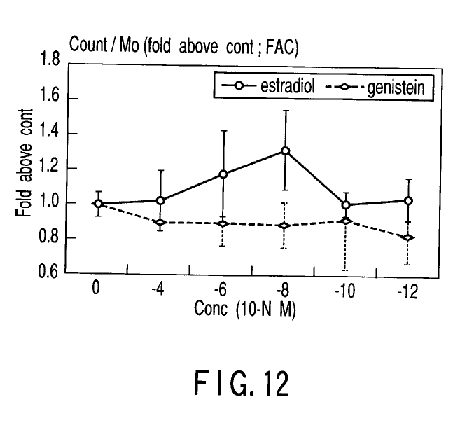

Zeiss Confocor was used as the device for analysis. Argon laser of 488 nm was irradiated for measuring the fusion protein of GFP and estrogen receptor, which had been produced according to the aforementioned procedure. A solution containing the fusion protein of GFP and estrogen receptor was prepared by diluting with phosphate buffer by 20 times. 5 μL of the prepared solution was dropped to “Labtech Chamber” (Nunc) and measured for 60 seconds using NDF (ND filter) of 1.5. The average value of “fluorescence intensity per molecule” (COUNT/MOL) of the five separate data was plotted to the graph 1 shown in FIG. 12, and the standard deviation thereof was expressed by dispersion (FIG. 12).

From the obtained result, it is understood that estradiol exhibits a strong binding force at the concentration of 10⁻⁸M and has sensitivity equal to that observed in the conventional hormonal action. Further, decrease in binding force at a high concentration is also observed, as is observed in the conventional e-SCREEN which uses cells, whereby it was proved that a signal reaction similar to that observed in cells occurs in the reproduced manner.

5) Method of binding protein to fluorescent beads

Even if a protein have been produced by using the expression vector without incorporating a fluorescent label thereto, the proteins can be adsorbed to fluorescent beads and the resultant proteins adsorbed to fluorescent beads can be analyzed by FCS technique, for the specific binding property effected by a test substance thereon.

Specifically, fluorescent beads whose particle diameter is in a range of 100 to 500 nm with little variation between particles and CV (coefficient of variation) is less then 3% should be used. Approximately 20 μL of a suspension of the fluorescent beads is thoroughly washed with a buffer by using a centrifuge. A few μg of a protein solution (such as a specific antibody or receptor) is mixed thereto and agitated in a buffer for 2 hours at 4° C., so that the protein is adsorbed to the fluorescent beads. The fluorescent beads are then collected by centrifuging. Centrifuging of the beads in a buffer (i.e., washing) are repeated four times, in order to remove the floating proteins. The eventually collected fluorescent beads have adsorbed the protein.

By using these fluorescent beads, the antigen specific to the antibody or a substance which is specifically bound to a receptor can be specifically detected by monomolecular photometry (e.g., fluorescence correlation spectroscopy).

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

“Sequence Listing” will be described on the next page and later.

What is claimed is:

1. A method of detecting presence/absence of binding capacity of a test substance, with respect to a protein, comprising:
(1) having a protein, which has been labeled with a fluorescence material, exist in a solution; and
(2) while successively measuring fluorescence intensity from the fluorescent material, reacting the test substance with the fluorescence-labeled protein described in (1) above and determining presence/absence of binding capacity of the test substance, with respect to the protein, on the basis of the successive change in fluorescence intensity.

2. The method of detecting presence/absence of binding capacity of a test substance, with respect to a protein according to claim 1, further comprising:
(3) determining presence/absence of binding capacity of the test substance with respect to the protein, on the basis of comparison of the successive change in fluorescence intensity obtained in (2) above with successive change in fluorescence intensity obtained in the absence of the test substance.

3. The detection method according to claim 1, wherein the step (1) comprises:
(a) constructing an expression vector by incorporating, to a vector, a gene encoding the protein, a gene encoding the fluorescent material for labeling the protein, and a promoter for expressing in vitro the gene encoding the protein; and
(b) having the expression vector exist in a solution which allows expression and transcription of the genes described in the step (a) and production of the protein, thereby producing the protein labeled with the fluorescent material.

4. The detection method according to claim 1, wherein the protein is selected from the group consisting of a hormone receptor, antigen and antibody.

5. The detection method according to claim 1, wherein the protein is an estrogen receptor.

6. The detection method according to claim 3, wherein the expression vector is as follows: the protein is estrogen receptor β, the fluorescent material for labeling is Green Fluorescent Protein, and the promoter is RNA polymerase promoter selected from the group consisting of T3, T7 and SP6.

7. The detection method according to claim 1, wherein the measurement and determination are carried out by the analysis according to Fluorescence Correlation Spectroscopy (FCS), Fluorescence Intensity Distribution Analysis (FIDA), or Fluorescence Intensity Multiple Distribution Analysis which effects FCS and FIDA at the same time.

8. A method of detecting a binding reaction of a test substance, to a receptor, comprising:
maintaining a receptor which has been labeled with a marker material capable of generating a light signal, a test substance, and a nucleic acid fragment having a specific nucleic acid sequence which allows binding of a receptor/ligand complex thereto, in a solution in which the receptor and the ligand thereof can form a complex and this receptor/ligand complex can be bound to the specific nucleic acid sequence which allows binding of the receptor/ligand complex thereto; and
detecting presence/absence of a complex constituted of the receptor, the test substance and the nucleic acid fragment, which is formed as a result of the receptor and the test substance forming a first complex and the first complex being bound to the nucleic acid fragment.

9. The method according to claim 8, wherein presence/absence of the receptor/test substance/nucleic acid fragment complex is detected by measuring diffusion time, in the solution, of the receptor labeled with a marker material capable of generating a light signal.

10. The method according to claim 8, wherein the receptor is a nuclear receptor.

11. The method according to claim 8, wherein the nucleic acid fragment having the specific nucleic acid sequence has molecular weight which is no smaller than that of the receptor.

12. The method according to claim 8, wherein the nucleic acid fragment having the specific nucleic acid sequence has diffusion constant which is no larger than that of the receptor.

13. The method according to claim 8, wherein binding affinity of the test substance with respect to the receptor is evaluated by expressing, through calculation using an autocorrelation function, the amount of the receptor monomer and the amount of the receptor/test substance/nucleic acid fragment complex with relative values.

14. The method according to claim 8, wherein the nucleic acid is DNA.

15. The method according to claim 8, wherein the detection is carried out according to high-throughput detection.

16. A method of producing a protein which has been labeled with fluorescence, comprising:
(a) constructing an expression vector by incorporating, to a vector, a gene encoding the protein, a gene encoding a fluorescent material for labeling the protein, and a promoter for expressing in vitro the gene encoding the protein; and
(b) having the expression vector exist in a solution which allows expression and transcription of the genes described in (a) above and production of the protein, thereby producing the protein labeled with the fluorescent material.

17. The method of producing a labeled protein according to claim 16, wherein the expression vector is as follows: the protein is estrogen receptor β, the fluorescent material for labeling is Green Fluorescent Protein, and the promoter is a RNA polymerase promoter selected from a group consisting of T3, T7 and SP6.

18. A labeled protein produced by the production method according to claim 16.


SEQUENCE LISTING (3 sequences)

SEQ ID NO: 1 - DNA, 1740 bp, Homo sapiens
  CDS (99)..(1688)
gttgacagccattatacttgcccacgaatctttgagaacattataatgacctttgtgcctcttcttgcaaggtgttttctcagctgttatctcaagacatggatataaaaaactcaccatctagccttaattctccttcctcctacaactgcagtcaatccatcttacccctggagcacggctccatatacataccttcctcctatgtagacagccaccatgaatatccagccatgacattctatagccctgctgtgatgaattacagcattcccagcaatgtcactaacttggaaggtgggcctggtcggcagaccacaagcccaaatgtgttgtggccaacacctgggcacctttctcctttagtggtccatcgccagttatcacatctgtatgcggaacctcaaaagagtccctggtgtgaagcaagatcgctagaacacaccttacctgtaaacagagagacactgaaaaggaaggttagtgggaaccgttgcgccagccctgttactggtccaggttcaaagagggatgctcacttctgcgctgtctgcagcgattacgcatcgggatatcactatggagtctggtcgtgtgaaggatgtaaggccttttttaaaagaagcattcaaggacataatgattatatttgtccagctacaaatcagtgtacaatcgataaaaaccggcgcaagagctgccaggcctgccgacttcggaagtgttacgaagtgggaatggtgaagtgtggctcccggagagagagatgtgggtaccgccttgtgcggagacagagaagtgccgacgagcagctgcactgtgccggcaaggccaagagaagtggcggccacgcgccccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagtaacaagggcatggaacatctgctcaacatgaagtgcaaaaatgtggtcccagtgtatgacctgctgctggagatgctgaatgcccacgtgcttcgcgggtgcaagtcctccatcacggggtccgagtgcagcccggcagaggacagtaaaagcaaagagggctcccagaacccacagtctcagtgacgcctggccctgaggtgaactggcccacagaggtcacaagctgaagcgt

SEQ ID NO: 2 - protein, 530 aa, Homo sapiens
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

SEQ ID NO: 3 - DNA, 15 bp, Artificial sequence
  misc_feature (7)..(7): n stands for any base
aggtcannntgacct